(19) United States
(12) Patent Application Publication
(10) Pub. No.: US 2025/0382565 A1
(43) Pub. Date: Dec. 18, 2025

(54) INTELLIGENT SUPERVISION SYSTEM AND METHOD FOR BACTERIAL CULTURE EQUIPMENT BASED ON BIG DATA

(71) Applicant: Suzhou Foo-Chi Biomedical Technology Co., Ltd., Suzhou (CN)

(72) Inventors: Xiang Li, Suzhou (CN); Ben Turng, Suzhou (CN); Yangguang Shao, Suzhou (CN)

(21) Appl. No.: 19/315,977

(22) Filed: Sep. 2, 2025

(30) Foreign Application Priority Data
Sep. 4, 2024 (CN) 202411231776.4

(51) Int. Cl. C12M 1/36; C12M 1/00; C12M 1/04; C12M 1/06; C12M 1/12; C12M 1/34

(52) U.S. Cl. CPC C12M 41/48; C12M 23/24; C12M 25/02; C12M 27/02; C12M 29/00; C12M 41/12; C12M 41/26; C12M 41/34

(57) ABSTRACT

An intelligent supervision system and method for bacterial culture equipment based on big data are provided, which relate to the technical field of bacterial culture. The system includes a host computer unit and a central control unit. The host computer unit is configured to generate and issue a control instruction. The central control unit is configured to: receive the control instruction issued by the host computer unit, control an execution unit to complete each control instruction, receive a feedback of a sensor bound to the execution unit, generate operating state data corresponding to the execution unit, and feed the operating state data back to the host computer unit. The system is convenient to operate, has high degree of automation, and can provide convenience for users.

### TECHNICAL FIELD

The present disclosure relates to the technical field of bacterial culture, and in particular, to an intelligent supervision system and method for bacterial culture equipment based on big data.

### BACKGROUND

Pharmacokinetics is a science that studies a time-dependent dynamic change law of the concentration of a drug after the drug enters a body, and pharmacodynamics is a science that mainly studies effects of a drug on pathogens and can reflect an antimicrobial effect and a clinical efficacy of the drug. In recent years, pharmacokinetics and pharmacodynamics have received increasing attention. Study data of in vitro PK/PD is essential for selecting doses and regimens for patients, establishing an antimicrobial susceptibility breakpoint, and ultimately perfecting clinical doses and regimens. At present, commonly used models for in vitro PK/PD include a static model, a dynamic model, and the like. A traditional bacterial culture system has a single function, a simple structure, and a low degree of automation, and cannot well meet diverse needs of users. The system is mainly dependent on manual operations, so that it is tedious and time-consuming, and the concentration of a culture medium in a bacterial culture process cannot be dynamically adjusted, which cannot guarantee the stability of a bacterial culture environment. As a result, PK/PD study data obtained based on bacterial culture has a significant deviation.

In the existing technology, Chinese patent CN110607344A discloses a construction method and use of a PK/PD model. An implementation process of the model is fully manual. Therefore, a constructed PK/PD model has advantages of high automation and high integration and can implement automation and integration functions, to ensure the stability of the bacterial culture environment in an experimental process.

### SUMMARY

The present disclosure aims to provide an intelligent supervision system and method for bacterial culture equipment based on big data, to solve the problems mentioned in the above background section.

In order to solve the above technical problems, the present disclosure provides the following technical solutions: an intelligent supervision system for bacterial culture equipment based on big data. The system includes a host computer unit and a central control unit.

The host computer unit is configured to generate and issue a control instruction.

The central control unit is configured to: receive the control instruction issued by the host computer unit, control an execution unit to complete each control instruction, receive a feedback of a sensor bound to the execution unit, generate operating state data corresponding to the execution unit, and feed the operating state data back to the host computer unit.

The execution unit includes a culture solution path unit, a filtering unit, and a sampling unit.

The culture solution path unit is configured to control a culture solution path in the bacterial culture equipment.

The filtering unit controls a filtering fiber in the bacterial culture equipment to recycle or filter a culture solution transferred from a culture bottle in the culture solution path; a filtrate flows back into the culture bottle, and recycled solution flows into a waste solution tank through a peristaltic pump.

The sampling unit is configured to control a sampling operation on the culture solution in the culture solution path of the bacterial culture equipment.

Further, the bacterial culture equipment includes the culture bottle and the filtering fiber. The culture bottle is connected to two ends of the filtering fiber through a solution path pipeline and a diaphragm pump to form a loop. The filtering fiber is further connected to the waste solution tank through the solution path pipeline and the diaphragm pump, to replace the culture solution in the culture bottle.

A magnetic stirrer is arranged inside the culture bottle. An electromagnet is arranged at a bottom of the culture bottle to drive the magnetic stirrer to rotate. The culture bottle is provided with a waterproof breathable film for controlling gas exchange between the culture bottle and the outside. A plurality of sensors are arranged in the culture bottle, to monitor state information inside the culture bottle.

Further, the culture solution path unit includes a control module, a detection module, and a solution adding module.

The control module is configured to control a temperature and a gas concentration inside the culture bottle.

In the present disclosure, temperature control is implemented through Peltier, and atmosphere control is implemented by connecting a gas valve and a sterile filter element to a gas bottle. During use, a suitable atmosphere (air, CO2, or nitrogen) can be directly added into a space above the culture bottle based on a requirement of an atmosphere for bacterial culture.

The detection module is configured to detect a liquid level height and PH value of the culture solution in the culture bottle. The liquid level height is monitored and obtained through a liquid level sensor arranged in the culture bottle. The PH value is determined by adding a preset indicator, and a color sensor identifies and controls a PH value of a solution in a corresponding culture bottle based on a color variable.

In the present disclosure, the liquid level height in the culture bottle is monitored because both the waste solution recycling operation and the sampling operation can reduce the volume of the culture medium in the culture bottle, which requires subsequent replenishment. The difference in the liquid level height is monitored to further determine the amount of the culture medium solution that needs to be replenished.

The solution adding module is configured to control a type and amount of a solution added into the culture bottle.

Further, when the filtering unit recycles the culture solution transferred from the culture bottle, the filtering unit generates a waste solution recycling monitored concentration threshold fluctuation disturbance variable corresponding to current time with reference to monitored information of the bacterial culture equipment in historical data. An obtaining method is as follows:
Step I: extracting, from the historical data, a culture solution concentration monitored by the sensor at each time point within a time period between two adjacent executions of a waste solution recycling operation, and constructing a culture solution concentration fluctuation data pair which is denoted as (t, Ct), where t represents a time interval from each time point within the time period between the two adjacent executions of the waste solution recycling operation and a time point of a previous execution of the waste solution recycling operation; Ct represents a corresponding culture solution concentration value when the time interval from each time point within the time period between the two adjacent executions of the waste solution recycling operation and the time point of the previous execution of the waste solution recycling operation is t; a value of Ct is equal to a reciprocal of 10 to the power of a PH value corresponding to t;
Step II: sequentially connecting corresponding coordinate points, in a rectangular plane coordinate system, of culture solution concentration fluctuation data pairs respectively corresponding to time points within a time period between any two adjacent executions of the waste solution recycling operation to obtain change relationship curves, corresponding to the corresponding two adjacent executions of the waste solution recycling operation, of culture solution concentrations and time, where the rectangular plane coordinate system is built by using o as an origin, using the time interval as an x axis, and using the culture solution concentrations as a y axis;
Step III: marking coordinate points, at which the culture solution concentrations are maximum, among corresponding coordinate points of the change relationship curves in the rectangular plane coordinate system at the same x-coordinate, and fitting the marked coordinate points in the rectangular plane coordinate system based on a preset function model y=, to generate a reference function of a change relationship between a culture solution concentration corresponding to the current time, and the time, the reference function being denoted as function (x);
Step IV: obtaining, from the historical data, an average value of an interval delay between generation of a waste solution recycling operation instruction and start of execution of the corresponding waste solution recycling operation instruction, the average value being denoted as TD, and calculating the waste solution recycling monitored concentration threshold fluctuation disturbance variable corresponding to the current time, denoted as AF, AF=(r+1)·function(TE−TD),
where TE represents a predicted value of a time interval between a time point of a next generation of a waste solution recycling operation instruction based on the current time and the time point of the previous execution of the waste solution recycling operation; a value of r is equal to an average value of data fitting deviations respectively corresponding to time points within a time period from the time point of the previous execution of the waste solution recycling operation based on the current time to the current time;
if a data fitting deviation corresponding to any time point is denoted as M, M=(Cs Cp)/Cp, where Cs represents a culture solution concentration actually monitored by the sensor at the corresponding time point, and Cp represents a culture solution concentration corresponding to a corresponding time point in function (x);

TE=function-1(AR/(r+1)),
where AR represents a preset waste solution recycling monitored concentration threshold set in a database in advance; function-1( ) represents an inverse function of function( ); function-1 (AR/(r+1)) represents a corresponding x-coordinate value when the culture solution concentration in function (x) is AR/(r+1); and r represents a function fitting deviation coefficient.

The filtering unit determines a generation condition of the waste solution recycling operation instruction based on the waste solution recycling monitored concentration threshold fluctuation disturbance variable AF corresponding to the current time.

When the sensor monitors that a concentration of the culture solution transferred from the culture bottle to the filtering fiber before recycling is less than or equal to AF, it is determined to immediately generate the waste solution recycling operation instruction; on the contrary, the filtering unit does not perform a recycling operation on the culture solution transferred from the culture bottle.

The waste solution recycling operation instruction includes waste solution recycling time and a waste solution recycling amount.

The waste solution recycling time is time at which the corresponding waste solution recycling operation instruction is generated; and the waste solution recycling amount is a manually set threshold.

Further, the solution adding module controls a type of a solution added into the culture bottle to be the same as a type of a culture medium solution selected for initial bacterial culture in a current culture bottle.

When the solution adding module controls an amount of the solution added into the culture bottle, a corresponding solution adding operation instruction includes solution adding operation time and a solution adding amount. The solution adding operation time is a time point at which a liquid level height fluctuation value monitored by the sensor within latest unit time is less than or equal to a preset fluctuation value and an absolute value of a difference between a corresponding liquid level height and a preset liquid level height is less than or equal to a preset liquid level deviation.

The solution adding amount is a culture solution volume corresponding to a liquid level height difference between a liquid level height corresponding to corresponding solution adding operation time and the preset liquid level height.

The unit time is a constant set in the database in advance; and the liquid level height fluctuation value is equal to a difference between a maximum liquid level height and a minimum liquid level height within a corresponding time period.

Further, the sampling unit performs a sampling operation on the culture solution once every preset time, and an amount of each sampling is set in advance.

An intelligent supervision method for bacterial culture equipment based on big data is provided. The method includes the following steps:
S1: setting operating parameters of the bacterial culture equipment through a host computer unit and a central control unit;
S2: receiving, through the central control unit, feedback data of a sensor bound to an execution unit corresponding to the bacterial culture equipment, generating operating state data corresponding to the execution unit, and feeding the operating state data back to the host computer unit; and
S3: generating, by the host computer unit based on the operating state data of the execution unit that is fed back by the central control unit and an operating parameter setting result corresponding to the bacterial culture equipment, a control instruction corresponding to the execution unit, and controlling the execution unit through the central control unit.

The control instruction includes an instruction for controlling recycling of a waste solution, an instruction for controlling a collection operation on a culture solution, an instruction for controlling an ambient temperature inside a culture bottle, an instruction for controlling an ambient atmosphere inside the culture bottle, an instruction for controlling update of a culture medium, and an instruction for controlling a temperature of a sample storage environment.

Further, the operating parameters include operating time of a culture solution path unit in the execution unit, the ambient temperature and ambient atmosphere inside the culture bottle, a volume and PH value of the culture medium, a condition of determining an update of the culture medium, a condition of recycling a waste solution in a filtering unit, a single recycling amount of the waste solution, and a sample storage temperature, sampling volume, and sampling frequency in a sampling unit.

Compared with the existing technology, the present disclosure achieves beneficial effects: The system is convenient to operate, has high degree of automation, and can provide convenience for users. The system provides a solution for in vitro study of a dynamic sterilization model, can simulate inhibition or killing effects of various different medication regimens on bacteria, and provides a reference for clinical medication. Meanwhile, in a simulation implementation process, a culture medium is dynamically updated to ensure stability of a bacterial culture environment, thus reducing a deviation in PK/PD study data obtained based on bacterial culture.

### BRIEF DESCRIPTION OF THE DRAWINGS

The accompanying drawings are intended to provide a further understanding of the present disclosure and constitute a part of this specification. The accompanying drawings and embodiments of the present disclosure are used together to explain the present disclosure rather than constituting a limitation on the present disclosure. In the accompanying drawings:

FIG. 1 is a schematic structural diagram of an intelligent supervision system for bacterial culture equipment based on big data according to the present disclosure.

### DETAILED DESCRIPTION OF THE EMBODIMENTS

The following clearly and completely describes the technical solutions in the embodiments of the present disclosure with reference to the accompanying drawings in the embodiments of the present disclosure. Apparently, the described embodiments are some of the embodiments of the present disclosure rather than all the embodiments. All other embodiments obtained by a person of ordinary skill in the art based on the embodiments of present disclosure without making creative efforts shall fall within the protection scope of present disclosure.

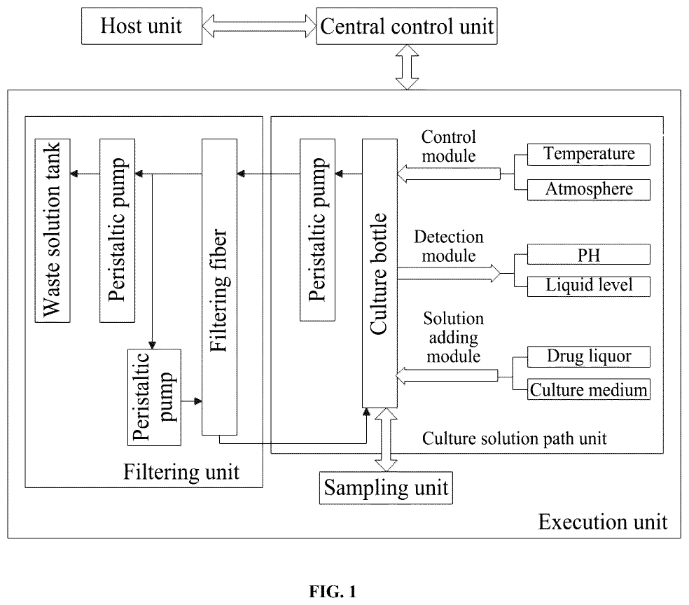

Referring to FIG. 1, the present disclosure provides a technical solution: an intelligent supervision method for bacterial culture equipment based on big data. The method includes the following steps:

S1: setting operating parameters of the bacterial culture equipment through a host computer unit and a central control unit. The operating parameters include operating time of a culture solution path unit in an execution unit, an ambient temperature and ambient atmosphere inside a culture bottle, a volume and PH value of a culture medium, a condition of determining an update of the culture medium, a condition of recycling a waste solution in a filtering unit, a single recycling amount of the waste solution, and a sample storage temperature, sampling volume, and sampling frequency in a sampling unit.

S2: receiving, through the central control unit, feedback data of a sensor bound to an execution unit corresponding to the bacterial culture equipment, generating operating state data corresponding to the execution unit, and feeding the operating state data back to the host computer unit.

S3: generating, by the host computer unit based on the operating state data of the execution unit that is fed back by the central control unit and an operating parameter setting result corresponding to the bacterial culture equipment, a control instruction corresponding to the execution unit, and controlling the execution unit through the central control unit.

The control instruction includes an instruction for controlling recycling of the waste solution, an instruction for controlling a collection operation on a culture solution, an instruction for controlling the ambient temperature inside the culture bottle, an instruction for controlling the ambient atmosphere inside the culture bottle, an instruction for controlling the update of the culture medium, and an instruction for controlling a temperature of a sample storage environment.

An intelligent supervision system for bacterial culture equipment based on big data is provided. The system includes a host computer unit and a central control unit.

The host computer unit is configured to generate and issue a control instruction.

The central control unit is configured to: receive the control instruction issued by the host computer unit, control an execution unit to complete each control instruction, receive a feedback of a sensor bound to the execution unit, generate operating state data corresponding to the execution unit, and feed the operating state data back to the host computer unit.

The execution unit includes a culture solution path unit, a filtering unit, and a sampling unit.

The filtering unit controls a filtering fiber in the bacterial culture equipment to recycle or filter a culture solution transferred from a culture bottle in the culture solution path; a filtrate flows back into the culture bottle, and recycled solution flows into a waste solution tank through a peristaltic pump.

The bacterial culture equipment includes the culture bottle and the filtering fiber. The culture bottle is connected to two ends of the filtering fiber through a solution path pipeline and a diaphragm pump to form a loop. The filtering fiber is further connected to the waste solution tank through the solution path pipeline and the diaphragm pump, to replace the culture solution in the culture bottle.

A magnetic stirrer is arranged inside the culture bottle. An electromagnet is arranged at a bottom of the culture bottle to drive the magnetic stirrer to rotate. The culture bottle is provided with a waterproof breathable film for controlling gas exchange between the culture bottle and the outside. A plurality of sensors are arranged in the culture bottle, to monitor state information inside the culture bottle.

When the filtering unit recycles the culture solution transferred from the culture bottle, the filtering unit generates a waste solution recycling monitored concentration threshold fluctuation disturbance variable corresponding to current time with reference to monitored information of the bacterial culture equipment in historical data. An obtaining method is as follows:
Step I: extracting, from the historical data, a culture solution concentration monitored by the sensor at each time point within a time period between two adjacent executions of a waste solution recycling operation, and constructing a culture solution concentration fluctuation data pair which is denoted as (t, Ct), where t represents a time interval from each time point within the time period between the two adjacent executions of the waste solution recycling operation and a time point of a previous execution of the waste solution recycling operation; Ct represents a corresponding culture solution concentration value when the time interval from each time point within the time period between the two adjacent executions of the waste solution recycling operation and the time point of the previous execution of the waste solution recycling operation is t; a value of Ct is equal to a reciprocal of 10 to the power of a PH value corresponding to t;
Step II: sequentially connecting corresponding coordinate points, in a rectangular plane coordinate system, of culture solution concentration fluctuation data pairs respectively corresponding to time points within a time period between any two adjacent executions of the waste solution recycling operation to obtain change relationship curves, corresponding to the corresponding two adjacent executions of the waste solution recycling operation, of culture solution concentrations and time, where the rectangular plane coordinate system is built by using o as an origin, using the time interval as an x axis, and using the culture solution concentrations as a y axis;
Step III: marking coordinate points, at which the culture solution concentrations are maximum, among corresponding coordinate points of the change relationship curves in the rectangular plane coordinate system at the same x-coordinate, and fitting the marked coordinate points in the rectangular plane coordinate system based on a preset function model y=, to generate a reference function of a change relationship between a culture solution concentration corresponding to the current time, and the time, the reference function being denoted as function (x);
Step IV: obtaining, from the historical data, an average value of an interval delay between generation of a waste solution recycling operation instruction and start of execution of the corresponding waste solution recycling operation instruction, the average value being denoted as TD, and calculating the waste solution recycling monitored concentration threshold fluctuation disturbance variable corresponding to the current time, denoted as AF,

$$AF = ( r + 1 ) \cdot function ( TE - TD ) ,$$

where TE represents a predicted value of a time interval between a time point of a next generation of a waste solution recycling operation instruction based on the current time and the time point of the previous execution of the waste solution recycling operation; a value of r is equal to an average value of data fitting deviations respectively corresponding to time points within a time period from the time point of the previous execution of the waste solution recycling operation based on the current time to the current time;
if a data fitting deviation corresponding to any time point is denoted as M, M=(Cs Cp)/Cp, where Cs represents a culture solution concentration actually monitored by the sensor at the corresponding time point, and Cp represents a culture solution concentration corresponding to a corresponding time point in function (x);

$$TE = function - 1 ( AR / ( r + 1 ) ) ,$$

where AR represents a preset waste solution recycling monitored concentration threshold set in a database in advance; function-1( ) represents an inverse function of function( ); function-1 (AR/(r+1)) represents a corresponding x-coordinate value when the culture solution concentration in function (x) is AR/(r+1); and r represents a function fitting deviation coefficient.

The filtering unit determines a generation condition of the waste solution recycling operation instruction based on the waste solution recycling monitored concentration threshold fluctuation disturbance variable AF corresponding to the current time.

When the sensor monitors that a concentration of the culture solution transferred from the culture bottle to the filtering fiber before recycling is less than or equal to AF, it is determined to immediately generate the waste solution recycling operation instruction; on the contrary, the filtering unit does not perform a recycling operation on the culture solution transferred from the culture bottle.

The waste solution recycling operation instruction includes waste solution recycling time and a waste solution recycling amount.

The waste solution recycling time is time at which the corresponding waste solution recycling operation instruction is generated; and the waste solution recycling amount is a manually set threshold.

The culture solution path unit is configured to control a culture solution path in the bacterial culture equipment.

The culture solution path unit includes a control module, a detection module, and a solution adding module.

The control module is configured to control a temperature and a gas concentration inside the culture bottle.

The detection module is configured to detect a liquid level height and PH value of the culture solution in the culture bottle. The liquid level height is monitored and obtained through a liquid level sensor arranged in the culture bottle. The PH value is determined by adding a preset indicator, and a color sensor identifies and controls a PH value of a solution in a corresponding culture bottle based on a color variable.

The solution adding module is configured to control a type and amount of a solution added into the culture bottle.

The solution adding module controls a type of a solution added into the culture bottle to be the same as a type of a culture medium solution selected for initial bacterial culture in a current culture bottle.

When the solution adding module controls an amount of the solution added into the culture bottle, a corresponding solution adding operation instruction includes solution adding operation time and a solution adding amount. The solution adding operation time is a time point at which a liquid level height fluctuation value monitored by the sensor within latest unit time is less than or equal to a preset fluctuation value and an absolute value of a difference between a corresponding liquid level height and a preset liquid level height is less than or equal to a preset liquid level deviation.

The solution adding amount is a culture solution volume corresponding to a liquid level height difference between a liquid level height corresponding to corresponding solution adding operation time and the preset liquid level height.

The unit time is a constant set in the database in advance; and the liquid level height fluctuation value is equal to a difference between a maximum liquid level height and a minimum liquid level height within a corresponding time period.

The sampling unit is configured to control a sampling operation on the culture solution in the culture solution path of the bacterial culture equipment.

The sampling unit performs a sampling operation on the culture solution once every preset time, and an amount of each sampling is set in advance.

This embodiment includes preparations before test:

Determination of a test plan: After investigation, cefuroxime belongs to a time-dependent antibacterial drug. Clinically, a medication regimen of administrating the drug for multiple times a day. In this test, the drug is administrated every eight hours, and each additional drug administration is completed within 15 minutes. A total test duration is 48 hours. Use of Staphylococcus aureus ATCC 29213 as a strain.

Test of the MIC of the antibiotic against the strain to be tested: The strain ATCC 29213 for test is incubated onto a culture medium plate, and a bacterial colony grows after 18 to 24 h for future use. Normal saline (a phosphate buffer solution, or the like) is prepared and is then separately put into turbidimeter tubes, each of which has a capacity of 9 mL, and sterilization treatment is carried out (at 121° C. for 15 to 30 min). A particular number of bacteria are added into the turbidimeter tubes to make bacterial suspensions, and the MacFarland turbidity is adjusted to 0.5 to 1 McFarland. According to a CLSI standard operation and use guidelines of E-test, a swab is dipped into each bacterial suspension, and is applied to a culture medium. After the swab is dried for 15 to 20 min, a cefuroxime E-test strip is applied to a surface of a bacterial moss for overnight culturing. A scale at an intersection of an edge of an inhibition zone and the strip is read. The scale is an MIC value of ATCC 29213 against the cefuroxime, which is 2 ug/mL.

Preparation of the fresh culture medium: MH broth is used as a culture medium. 21 g of MH broth dry powder (Sigma) is weighed and dissolved in 1 L distilled water. High-pressure sterilization is carried out at 121° C. for 15 min. The broth is then cooled for use.

Preparation of the antibacterial drug mother liquor: The antibacterial drug mother liquor is prepared by using cefuroxime axetil sodium for injection (1.5 g/bottle); 150 mL of the normal saline is used to dissolve one bottle of medicament, and a concentration of the cefuroxime is 10 mg/mL. The prepared antibacterial drug mother liquor is ready for later use.

Preparation of the bacterial suspension: The normal saline (the phosphate buffer solution or the like) is separately put into test tubes, each of which has a capacity of 9 mL; sterilization treatment is carried out (at 121° C. for 15 to 30 min). The sterilized test tubes with 9 mL of normal saline are numbered with 1, 2, 3, 4, 5, 6, 7, and 8. A particular number of bacteria is added into test tube 1 and is mixed uniformly with a vortex oscillator. The McFarland turbidity is adjusted to 0.5 McFarland, to obtain a bacterial suspension with a first dilution gradient and a bacterial count of approximately 1.5×108 CFU/mL. One mL of the bacterial suspension with the first gradient is drawn into test tube 2 and is mixed uniformly to obtain a bacterial suspension with a second dilution gradient, and so on.

Through the host computer unit and the central control unit, a program of the device is set, and parameters mainly include: operating time, temperature control, atmosphere control, a volume of the culture medium in the culture bottle, power (i.e. a flow rate) of a plurality of peristaltic pumps, PH value detection, a time interval and other threshold conditions for updating the culture medium/drug solution, a temperature, sampling volume, and sampling frequency of a sample storage region, and the like.

Dynamic adjustment of a bacterial culture environment:
Operating time: 48 h;
Temperature setting: 35 to 37° C.;
Atmosphere control: set an air atmosphere according to a suitable environment for the growth of the Staphylococcus aureus;
Volume of the culture medium in the culture bottle: set it to 200 mL. There is the liquid level sensor in the culture bottle. Based on a signal of the sensor, the central control unit can calculate a volume of a solution in the culture bottle;
Power (i.e. flow rate) of the plurality of peristaltic pumps: set it to 30 mL/min;
PH value: after investigation, a suitable PH value for culturing the Staphylococcus aureus is 7.2 to 7.4. A boundary value is set to 6.5 and 8.5. If a PH reaches the boundary value, PH adjustment will be initiated.

Time interval for updating the drug solution: after investigation, a recommended time interval for clinical intravenous infusion of cefuroxime is 8 h, so the time interval is set to 8 h;
Volume of the culture medium for replacement: a culture medium replacement threshold (a waste solution recycling amount) is 40 mL, and replacement time of the culture medium is dynamically adjusted;
Temperature of the sample storage region: 4° C.;
Sampling frequency and volume: set the sampling frequency to 90 min, and sample 2 mL at each time.

After the settings are completed, preheating of the system is completed after 30 to 60 min.

### Test Process:

According to the program of the operating time that is set in advance, the central control unit can stop the test within 48 h of set stop time.

According to the temperature parameter set in advance, the central control unit controls the power of Peltier to maintain the temperature of the culture bottle at the preset temperature of 35 to 37° C., with an error not exceeding ±2° C.

According to the atmosphere parameter set in advance, the central control unit controls an inlet valve to maintain the atmosphere above the culture bottle to be the preset air atmosphere.

According to the power of the peristaltic pumps that is set in advance, the central control unit can control the peristaltic pumps to provide power for flowing of liquid in the system within the set time period based on the preset power and flow rate.

According to the PH threshold set in advance, if the PH in the culture bottle reaches the boundary value, the PH adjustment will be initiated. This is implemented by updating the culture medium.

In the test process, the culture medium can be updated through the central control unit. As the volume of the culture medium solution for replacement is set to 40 mL in advance, the central control unit can discharge 40 mL of the culture medium into the waste solution tank and inject fresh drug-containing culture medium into the culture bottle. The signal fed back by the liquid level sensor can be used to determine a liquid level.

According to the sampling frequency and the sampling volume that are set in advance, a sampling unit can carry out sampling every 90 min at a sampling volume of 2 mL and store a sample in the sample storage region at 4° C. A user can carry out further analysis on the sample, such as drug concentration analysis, inoculation culture to observe a bacterial count, and flow cytometry analysis.

The test is completed:

Based on analysis results of the previous step, PD parameters can be obtained.

It should be noted that in this document, relationship terms such as first and second are used solely to distinguish one entity or operation from another entity or operation without necessarily requiring or implying any actual such relationship or order between such entities or operations. Furthermore, the terms “include”, “contain”, or any other variation thereof, are intended to encompass a non-exclusive inclusion, such that a process, method, article, or device that includes a list of elements does not include only those elements but may include other elements not explicitly listed or inherent to such process, method, article, or device.

It should be finally noted that: The foregoing embodiments are merely preferred embodiments of the present disclosure, but not intended to limit the present disclosure. Although the present disclosure is described in detail with reference to the foregoing embodiments, a person of ordinary skill in the art may still make modifications to the technical solutions described in the foregoing respective embodiments or make equivalent replacements to partial technical features thereof. Any modification, equivalent replacement, and improvement made within the spirit and scope of the present disclosure shall fall within the protection scope of the present disclosure.

What is claimed is:

1. An intelligent supervision system for bacterial culture equipment based on big data, wherein the system comprises:
a host computer unit, wherein the host computer unit is configured to generate and issue a control instruction; and
a central control unit, wherein the central control unit is configured to: receive the control instruction issued by the host computer unit, control an execution unit to complete each control instruction, receive a feedback of a sensor bound to the execution unit, generate operating state data corresponding to the execution unit, and feed the operating state data back to the host computer unit;
the execution unit comprises a culture solution path unit, a filtering unit, and a sampling unit;
the culture solution path unit is configured to control a culture solution path in the bacterial culture equipment;
the filtering unit controls a filtering fiber in the bacterial culture equipment to recycle or filter a culture solution transferred from a culture bottle in the culture solution path; a filtrate flows back into the culture bottle, and recycled solution flows into a waste solution tank through a peristaltic pump; and
the sampling unit is configured to control a sampling operation on the culture solution in the culture solution path of the bacterial culture equipment.

2. The intelligent supervision system for the bacterial culture equipment based on the big data according to claim 1, wherein the bacterial culture equipment comprises the culture bottle and the filtering fiber; the culture bottle is connected to two ends of the filtering fiber through a solution path pipeline and a diaphragm pump to form a loop; the filtering fiber is further connected to the waste solution tank through the solution path pipeline and the diaphragm pump, to replace the culture solution in the culture bottle;
a magnetic stirrer is arranged inside the culture bottle; an electromagnet is arranged at a bottom of the culture bottle to drive the magnetic stirrer to rotate; the culture bottle is provided with a waterproof breathable film for controlling gas exchange between the culture bottle and the outside; and a plurality of sensors are arranged in the culture bottle, to monitor state information inside the culture bottle.

3. The intelligent supervision system for the bacterial culture equipment based on the big data according to claim 1, wherein the culture solution path unit comprises a control module, a detection module, and a solution adding module;
the control module is configured to control a temperature and a gas concentration inside the culture bottle;
the detection module is configured to detect a liquid level height and PH value of the culture solution in the culture bottle; the liquid level height is monitored and obtained through a liquid level sensor arranged in the culture bottle; the PH value is determined by adding a preset indicator, and a color sensor identifies and controls a PH value of a solution in a corresponding culture bottle based on a color variable; and
the solution adding module is configured to control a type and amount of a solution added into the culture bottle.

4. The intelligent supervision system for the bacterial culture equipment based on the big data according to claim 1, wherein when the filtering unit recycles the culture solution transferred from the culture bottle, the filtering unit generates a waste solution recycling monitored concentration threshold fluctuation disturbance variable corresponding to current time with reference to monitored information of the bacterial culture equipment in historical data; and an obtaining method is as follows:
Step I: extracting, from the historical data, a culture solution concentration monitored by the sensor at each time point within a time period between two adjacent executions of a waste solution recycling operation, and constructing a culture solution concentration fluctuation data pair which is denoted as (t, Ct), wherein t represents a time interval from each time point within the time period between the two adjacent executions of the waste solution recycling operation and a time point of a previous execution of the waste solution recycling operation; Ct represents a corresponding culture solution concentration value when the time interval from each time point within the time period between the two adjacent executions of the waste solution recycling operation and the time point of the previous execution of the waste solution recycling operation is t; a value of Ct is equal to a reciprocal of 10 to the power of a PH value corresponding to t;
Step II: sequentially connecting corresponding coordinate points, in a rectangular plane coordinate system, of culture solution concentration fluctuation data pairs respectively corresponding to time points within a time period between any two adjacent executions of the waste solution recycling operation to obtain change relationship curves, corresponding to the corresponding two adjacent executions of the waste solution recycling operation, of culture solution concentrations and time, wherein the rectangular plane coordinate system is built by using o as an origin, using the time interval as an x axis, and using the culture solution concentrations as a y axis;
Step III: marking coordinate points, at which the culture solution concentrations are maximum, among corresponding coordinate points of the change relationship curves in the rectangular plane coordinate system at the same x-coordinate, and fitting the marked coordinate points in the rectangular plane coordinate system based on a preset function model y=, to generate a reference function of a change relationship between a culture solution concentration corresponding to the current time, and the time, the reference function being denoted as function (x);
Step IV: obtaining, from the historical data, an average value of an interval delay between generation of a waste solution recycling operation instruction and start of execution of the corresponding waste solution recycling operation instruction, the average value being denoted as TD, and calculating the waste solution recycling monitored concentration threshold fluctuation disturbance variable corresponding to the current time, denoted as AF,
$$AF = ( r + 1 ) \cdot function ( TE - TD ) ,$$
wherein TE represents a predicted value of a time interval between a time point of a next generation of a waste solution recycling operation instruction based on the current time and the time point of the previous execution of the waste solution recycling operation; a value of r is equal to an average value of data fitting deviations respectively corresponding to time points within a time period from the time point of the previous execution of the waste solution recycling operation based on the current time to the current time;
if a data fitting deviation corresponding to any time point is denoted as M, M=(Cs Cp)/Cp, wherein Cs represents a culture solution concentration actually monitored by the sensor at the corresponding time point, and Cp represents a culture solution concentration corresponding to a corresponding time point in function (x);
$$TE = function - 1 ( AR / ( r + 1 ) ) ,$$
wherein AR represents a preset waste solution recycling monitored concentration threshold set in a database in advance; function-1( ) represents an inverse function of function( ); function-1 (AR/(r+1)) represents a corresponding x-coordinate value when the culture solution concentration in function (x) is AR/(r+1); r represents a function fitting deviation coefficient;
the filtering unit determines a generation condition of the waste solution recycling operation instruction based on the waste solution recycling monitored concentration threshold fluctuation disturbance variable AF corresponding to the current time;
when the sensor monitors that a concentration of the culture solution transferred from the culture bottle to the filtering fiber before recycling is less than or equal to AF, it is determined to immediately generate the waste solution recycling operation instruction; on the contrary, the filtering unit does not perform a recycling operation on the culture solution transferred from the culture bottle;
the waste solution recycling operation instruction comprises waste solution recycling time and a waste solution recycling amount;
the waste solution recycling time is time at which the corresponding waste solution recycling operation instruction is generated; and the waste solution recycling amount is a manually set threshold.

5. The intelligent supervision system for the bacterial culture equipment based on the big data according to claim 4, wherein the solution adding module controls a type of a solution added into the culture bottle to be the same as a type of a culture medium solution selected for initial bacterial culture in a current culture bottle;
when the solution adding module controls an amount of the solution added into the culture bottle, a corresponding solution adding operation instruction comprises solution adding operation time and a solution adding amount; the solution adding operation time is a time point at which a liquid level height fluctuation value monitored by the sensor within latest unit time is less than or equal to a preset fluctuation value and an absolute value of a difference between a corresponding liquid level height and a preset liquid level height is less than or equal to a preset liquid level deviation;
the solution adding amount is a culture solution volume corresponding to a liquid level height difference between a liquid level height corresponding to corresponding solution adding operation time and the preset liquid level height;
the unit time is a constant set in the database in advance; and the liquid level height fluctuation value is equal to a difference between a maximum liquid level height and a minimum liquid level height within a corresponding time period.

6. The intelligent supervision system for the bacterial culture equipment based on the big data according to claim 5, wherein the sampling unit performs a sampling operation on the culture solution once every preset time, and an amount of each sampling is set in advance.

7. An intelligent supervision method for bacterial culture equipment based on big data, wherein the method comprises the following steps:
S1: setting operating parameters of the bacterial culture equipment through a host computer unit and a central control unit;
S2: receiving, through the central control unit, feedback data of a sensor bound to an execution unit corresponding to the bacterial culture equipment, generating operating state data corresponding to the execution unit, and feeding the operating state data back to the host computer unit; and
S3: generating, by the host computer unit based on the operating state data of the execution unit that is fed back by the central control unit and an operating parameter setting result corresponding to the bacterial culture equipment, a control instruction corresponding to the execution unit, and controlling the execution unit through the central control unit;
the control instruction comprises an instruction for controlling recycling of a waste solution, an instruction for controlling a collection operation on a culture solution, an instruction for controlling an ambient temperature inside a culture bottle, an instruction for controlling an ambient atmosphere inside the culture bottle, an instruction for controlling an update of a culture medium, and an instruction for controlling a temperature of a sample storage environment.

8. The intelligent supervision method for the bacterial culture equipment based on the big data according to claim 7, wherein the operating parameters comprise operating time of a culture solution path unit in the execution unit, the ambient temperature and ambient atmosphere inside the culture bottle, a volume and PH value of the culture medium, a condition of determining the update of the culture medium, a condition of recycling a waste solution in a filtering unit, a single recycling amount of the waste solution, and a sample storage temperature, sampling volume, and sampling frequency in a sampling unit.
